(19) United States
(12) Patent Application Publication
(10) Pub. No.: US 2007/0166273 A1
(43) Pub. Date: Jul. 19, 2007

(54) Skin treatment educational kit

(76) Inventors: Joseph J. Krivulka, (US); Leonard L. Mazur, (US)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 11/418,514

(22) Filed: May 4, 2006

Related U.S. Application Data
(60) Provisional application No. 60/760,121, filed on Jan. 19, 2006.

(51) Int. Cl. A61K 8/97; A61K 36/82; A61K 36/886

(52) U.S. Cl. 424/74; 424/729; 424/744

(57) ABSTRACT

A method to ameliorate the skin-irritating effects of topical tretinoin treatment by providing the tretinoin-using patient with a skin-care kit which includes (1) topical tretinoin; and (2) a skin cleanser formulated to minimize tretinoin-induced skin irritation, and (3) a skin moisturizer formulated to reduce tretinoin-induced skin irritation; and (4) packaging to present the aforementioned components together as a unified system.

### RELATED APPLICATIONS

This application claims priority from provisional filing Ser. No. 60/760,121, filed 19 Jan. 2006, the contents of which are here included by reference.

### GOVERNMENT INTEREST

None

### BACKGROUND

Acne vulgaris is a multifactorial skin disease that involves several processes:
Androgenic hormonal stimulation of the sebaceous glands, and abnormal desquamation of follicular keratinocytes in the pilosebaceous duct, leading to formation of microcomedones.
Excessive production of sebum.
Proliferation of P. acnes (Propionibacterium acnes) and follicular inflammation processes.
Production of inflammatory-inducing compounds (partially caused by the P. acnes population within the follicle), most notably neutrophil chemoattractants.
Changes in the permeability of the follicle wall, allowing release of bacterial antigens and inflammatory mediators, which drive the shift from non-inflammatory to inflammatory acne lesions.
   The literature suggests that a sound understanding of the pathophysiology of acne is key in determining optimal treatment. Therefore, appropriate, effective treatment will target:
normalization of follicular keratinization.
reduction of interfollicular P. acnes.
reduction of inflammation.
reduction of sebaceous gland activity.

Numerous topical medications are available for acne treatment, including retinoids and retinoid-like drugs, benzoyl peroxide, and antibiotics. Relatively less severe cases of acne can frequently be treated effectively with topical agents only. To avoid systemic toxicity, topicals are generally preferred to systemic therapy if favorable results can be maintained. In more severe cases, however, oral retinoids (e.g., isotretinoin) and oral antibiotics are commonly prescribed.

The topical retinoids, which include tretinoin (all-trans retinoic acid), adapalene, and tazarotene are the most frequently used topical medications. Topical retinoids are, in fact, considered to be a “mainstay” for effective and safe treatment of acne. Among the topical retinoids, tretinoin in particular has had a long and successful history of use. In 1969, it was the first retinoid shown to be effective in acne treatment⁹, and in 1971 was introduced in the US.

The basis for the effectiveness of topical tretinoin, and its status for the past several decades as a treatment mainstay, is its mechanism of action. As a retinoid, topical tretinoin acts on key patho-physiological processes of acne:
It counteracts the abnormal desquamation of follicular keratinocytes, leading to significant reductions in inflammatory lesion counts. Historically, dermatologists prescribed it heavily for patients with a predominance of non-inflammatory lesions.
It also reduces inflammation, as shown in clinical trials that have reported significant decreases in inflammatory lesions.
Normalization of desquamation, coupled with a reduction in inflammation, leads to a decrease in existing comedones, as well as inhibition of new comedones (the precursors of acne lesions).
   To summarize, topical tretinoin has a long history of use, and is considered effective and safe. As a result, it is a “mainstay” of therapy, and a standard, often first-line treatment for mild to moderate non-inflammatory acne, as well as for inflammatory acne.

Tretinoin is a dermatological pharmaceutical. It is available by prescription in the United States of America. It is known to be useful to treat various dermatological conditions. It is known to be useful topically.

One of the drawbacks of the topical use of tretinoin, however, is that topical usage is known in the art to irritate the skin. This propensity of tretinoin to irritate the skin makes its topical use a somewhat less attractive therapeutic alternative, for a number of reasons. For example, physicians who prescribe topical tretinoin may need to face up to patients who are irritated and dissatisfied. These patients may in turn refuse to comply with the recommended treatment regimen, due to such irritation. Such patient non-compliance would be expected to result in less-than-optimal clinical outcomes.

We have discovered a solution which may improve patient compliance and thus improve clinical outcomes, and decrease skin irritation and thereby increase the patient's satisfaction with both the tretinoin treatment itself and with the prescribing physician. Our solution involves providing the patient with the a complete skin-care kit, which includes (1) a topical tretinoin medication, and (2) a skin cleanser formulated to minimize tretinoin-induced skin irritation, and (3) a skin moisturizer formulated to reduce tretinoin-induced skin irritation, and (4) a presentation package to present all three of the foregoing components to the user as a unit, to psychologically reinforce the desirability of using all three of these components together.

### DETAILED DESCRIPTION

Our invention is a kit which includes four items: (1) a topical tretinoin medication, and (2) a skin cleanser formulated to minimize tretinoin-induced skin irritation, (3) a skin moisturizer formulated to reduce tretinoin-induced skin irritation, and (4) a presentation package to present all three of the foregoing components to the user as a unit. We discuss each in turn.

Topical Tretinoin

Topical tretionoin may be formulated as a gel, cream, liquid, et cetera. It is used for the topical treatment of acne vulgaris. We prefer that each gram contain from about 0.025 to 0.01% tretinoin. We prefer the gel include hydroxypropyl cellulose, butylated hydroxytoluene, and alcohol (denatured with tert-butyl alcohol and brucine sulfate 90% w/w). We prefer a cream to be a hydrophilic cream vehicle of (in order of relative amount) stearic acid, isopropyl myristate, polyoxyl 40 stearate, stearyl alcohol, xanthan gum, sorbic acid, butylated hydroxytoluene, and purified water.

Chemically, tretinoin is all-trans-retinoic acid. It has a molecular weight of 300.44 and has the following structural formula:

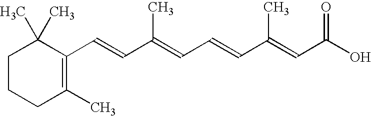

Although the exact mode of action of tretionoin is unknown, current evidence suggests that topical tretinoin decreases cohesiveness of follicular epithelial cells with decreased microcomedo formation. Additionally, tretinoin stimulates mitotic activity and increased turnover of follicular epithelial cells causing extrusion of the comedones.

Tretionoin gel and cream are indicated for topical application in the treatment of acne vulgaris. The safety and efficacy of the long-term use of this product in the treatment of other disorders have not been established. Use of tretionoin should be discontinued if hypersensitivity to any of the ingredients is noted.

If a reaction suggesting sensitivity or chemical irritation occurs, use of the medication should be discontinued. Exposure to sunlight, including sunlamps, should be minimized during the use of tretionoin, and patients with sunburn should be advised not to use the product until fully recovered because of heightened susceptibility to sunlight as a result of the use of tretinoin. Patients who may be required to have considerable sun exposure due to occupation and those with inherent sensitivity to the sun should exercise particular caution. Use of sunscreen products and protective clothing over treated areas is recommended when exposure cannot be avoided. Weather extremes, such as wind or cold, also may be irritating to patients under treatment with tretinoin.

Tretionoin preparations for acne treatment should be kept away from the eyes, the mouth, angles of the nose, and mucous membranes. Topical use may induce severe local erythema and peeling at the site of application. If the degree of local irritation warrants, patients should be directed to use the medication less frequently, discontinue use temporarily, or discontinue use altogether. Tretinoin has been reported to cause severe irritation on eczematous skin and should be used with utmost caution in patients with this condition.

Concomitant topical medication, medicated or abrasive soaps and cleansers, soaps and cosmetics that have a strong drying effect, and products with high concentrations of alcohol, astringents, spices or lime should be used with caution because of possible interaction with tretinoin. Particular caution should be exercised in using preparations containing sulfur, resorcinol, or salicylic acid with tretionoin. It also is advisable to “rest” a patient's skin until the effects of such preparations subside before use of tretionoin is begun.

Carcinogenesis, Mutagenesis, Impairment to Fertility: In a 91-week dermal study in which CD-1 mice were administered 0.017% and 0.035% formulations of tretinoin, cutaneous squamous cell carcinomas and papillomas in the treatment area were observed in some female mice. A dose-related incidence of liver tumors in male mice was observed at those same doses. The maximum systemic doses associated with the administered 0.017% and 0.035% formulations are 0.5 and 1.0 mg/kg/day, respectively. These doses are two and four times the maximum human systemic dose, when adjusted for total body surface area.

The biological significance of these findings is not clear because they occurred at doses that exceeded the dermal maximally tolerated dose (MTD) of tretinoin and because they were within the background natural occurrence rate for these tumors in this strain of mice. There was no evidence of carcinogenic potential when 0.025 mg/kg/day of tretinoin was administered topically to mice (0.1 times the maximum human systemic dose, adjusted for total body surface area). For purposes of comparisons of the animal exposure to systemic human exposure, the maximum human systemic dose is defined as 1 gram of 0.1% TRETIN∘X applied daily to a 50 kg person (0.02 mg tretinoin/kg body weight).

Studies in hairless albino mice suggest that concurrent exposure to tretinoin may enhance the tumorigenic potential of carcinogenic doses of UVB and UVA light form a solar simulator. This effect has been confirmed in a later study in pigmented mice, and dark pigmentation did not overcome the enhancement of photocarcinogenesis by 0.05% tretinoin. Although the significance of these studies to humans is not clear, patients should minimize exposure to sunlight or artificial ultraviolet irradiation sources.

The mutagenic potential of tretinoin was evaluated in the Ames assay and in the in vivo mouse micronucleus assay, both of which were negative.

In dermal Segment I fertility studies of tretinoin in rats, slight (not statistically significant) decreases in sperm count and motility were seen at 0.5 mg/kg/day (4 times the maximum human systemic dose adjusted for total body surface area), and slight (not statistically significant) increases in the number and percent of nonviable embryos in females treated with 0.25 mg/kg/day (2 times the maximum human systemic dose adjusted for total body surface area) and above were observed. A dermal Segment III study with tretionoin has not been performed in any species. In oral Segment I and Segment III studies in rats with tretinoin, decreased survival of neonates and growth retardation were observed at doses in excess of 2 mg/kg/day (16 times the human topical dose adjusted for total body surface area).

Oral tretinoin has been shown to be teratogenic in rats, mice, hamsters, and subhuman primates. It was teratogenic and fetotoxic in Wistar rats when given orally or topically in doses greater than 1 mg/kg/day (8 times the maximum human systemic dose adjusted for total body surface area). However, variations in teratogenic doses among various strains of rats have been reported. In the cynomolgus monkey, which metabolically is closer to humans for tretinoin than the other species examined, fetal malformations were reported at doses of 10 mg/kg/day or greater, but none were observed at 5 mg/kg/day (83 times the maximum human systemic dose adjusted for total body surface area), although increased skeletal variations were observed at all doses. A dose-related increase in embryolethality and abortion was reported. Similar results have also been reported in pigtail macaques.

Topical tretinoin in animal teratogenicity tests has generated equivocal results. There is evidence for teratogenicity (shortened or kinked tail) of topical tretinoin in Wistar rats at doses greater than 1 mg/kg/day (8 times the maximum human systemic dose adjusted for total body surface area). Anomalies (humerus: short 13%, bent 6%, os parietal incompletely ossified 14%) have also been reported when 10 mg/kg/day was topically applied.

There are other reports in New Zealand White rabbits administered doses of greater than 0.2 mg/kg/day (3.3 times the maximum human systemic dose adjusted for total body surface area) of an increased incidence of domed head and hydrocephaly, typical of retinoid-induced fetal malformations in this species.

In contrast, several well-controlled animal studies have shown that dermally applied tretinoin may be fetotoxic, but not overly teratogenic in tats and rabbits at doses of 1.0 and 0.5 mg/kg/day, respectively (8 times the maximum human systemic dose adjusted for total body surface area in both species).

With widespread use of any drug, a small number of birth defect reports associated temporally with the administration of the drug would be expected by chance alone. Thirty human cases of temporally associated congenital malformations have been reported during two decades of clinical use of tretinoin. Although no definite pattern of teratogenicity and no causal association has been established from these cases, five of the reports describe the rare birth defect category holoprosencephaly (defects associated with incomplete midline development of the forebrain). The significance of these spontaneous reports in terms of risk to the fetus is not known.

Topical tretinoin has been shown to be fetotoxic in rabbits when administered 0.5 mg/kg/day (8 times the maximum human systemic dose adjusted for total body surface area). Oral tretinoin has been shown to be fetotoxic, resulting in skeletal variations and increased intrauterine death in rats when administered 2.5 mg/kg/day (20 times the maximum human systemic dose adjusted for total body surface area).

There are no adequate and well-controlled studies in pregnant women. Tretinoin should be used during pregnancy only if the potential benefit justifies the potential risk to the fetus. It is not known whether tretionoin is excreted in human milk. Because many drugs are excreted in human milk, caution should be exercised when tretionoin is used by a nursing woman. Safety and effectiveness in pediatric patients below the age of 12 have not been established. Safety and effectiveness in a geriatric population have not been established. Clinical studies of tretinoin did not include sufficient numbers of subjects aged 65 and over to determine whether they respond differently from younger patients.

It is known in the art that with tretinoin use, the skin of certain sensitive individuals may become excessively red, edematous, blistered, or crusted. If these effects occur, the tretionoin use should either be discontinued until the integrity of the skin is restored, or the medication should be adjusted to a level the patient can tolerate. True contact allergy to topical tretinoin is rarely encountered. Temporary hyper- or hypopigmentation has been reported with repeated application of a tretinoin preparation. Some individuals have been reported to have heightened susceptibility to sunlight while under treatment with tretinoin. To date, all adverse effects of tretinoin have been reversible upon discontinuance of therapy (see Dosage and Administration Section).

Similarly, if medication is applied excessively, no more rapid or better results will be obtained and marked redness, peeling, or discomfort may occur. Oral ingestion of the drug may lead to the same side effects as those associated with excessive oral intake of Vitamin A.

We prefer that tretionoin gel or cream be applied once a day, before retiring, to the skin where acne lesions appear, using enough to cover the entire affected area lightly. For a gel formulation, excessive application results in “pilling” of the gel, which minimizes the likelihood of over-application by the patient. Application may cause a transitory feeling of warmth or slight stinging. In cases where it has been necessary to temporarily discontinue therapy or to reduce the frequency of application, therapy may be resumed or frequency of application increased when the patients become able to tolerate the treatment.

During the early weeks of therapy, an apparent exacerbation of inflammatory lesions may occur. This is due to the action of the medication on deep, previously unseen lesions and should not be considered a reason to discontinue therapy.

Therapeutic results should be noticed after two to three weeks but more than six weeks of therapy may be required before definite beneficial effects are seen. Once the acne lesions have responded satisfactorily, it may be possible to maintain the improvement with less frequent applications, or other dosage forms.

Patients treated with tretionoin preparations may use cosmetics, but the areas to be treated should be cleansed thoroughly before the medication is applied.

Skin Cleanser

We prefer the skin cleanser to be a gentle, non-soap formulation to avoid drying the skin. We prefer this formula to be made of a base of water and sodium laureth sulfate. To this base, we add cocamidopropyl betaine, cocamide MEA, polyquaternium-7, PEG-12, dimethicone, disodium cocamphodiacetate, panthenol, PEG-150 distearate, coenzyme Q-10 (ubiquinone), phenoxyethanol, sodium chloride, methylparaben, propylparaben, citric acid and disodium EDTA. This provides a foaming cleanser which gently cleans the skin.

We prefer the skin cleanser to include components to sooth the skin. For example, we prefer to include green tea (Camellia sinensis) extract.

The cleanser may be buffered to an appropriate pH to minimize the likelihood of skin irritation. We prefer that the cleanser have no added perfumes, to minimize the possibility that the cleanser will exacerbate tretinoin-caused dermal irritation.

Skin Moisturizer

After the patient uses the skin cleanser, we prefer the patient to then use a skin moisturizer which is light, non-greasy and soothing. We prefer to use a moisturizing base made of water and cetearyl alcohol.

We prefer to include PPG-2 myristyl ether propionate, squalane, dimethicone, polysorbate-60, polysorbate-20, hydroxycellulose, carbomer, butylene glycol, laureth-3, ethylene brassylate, beeswax, triethanolamine, methylparaben, propylparaben, imidazolidinyl urea, benzyl alcohol and disodium EDTA.

We prefer to include components which sooth skin irritation; these include Aloe barbadensis leaf juice (aloe vera gel), glycerine, green tea (Camellia sinensis) extract, acetyl dipeptide-1 cetyl ester and bisabolol.

Packaging

As we discuss above, tretinoin topical may cause skin irritation. Thus, it may be possible to simply advise the patient to use trial-and-error to test various skin cleansers to identify a gentle skin cleanser which does not exacerbate tretinoin-associated skin irritation. This approach, however, entails several shortcomings.

First, a trial-and-error process will very likely force the patient to use several cleansers which exacerbate the tretinoin-associated skin irritation; this greatly increases the risk of patient dissatisfaction and patient “non-compliance” (that is, a patient ceasing to use, and refusing to further take the medication at all, or refusing to take the medication with the clinically-desired frequency).

Second, this risk is especially great where, as here, the benefits of the medication are not seen until after some delay. With tretinoin, visible benefit usually occurs after six weeks of treatment. At the early stages of tretinoin treatment, tretinoin may actually cause a skin condition which to the patient looks like a worsening of acne vulgaris, rather than an improvement of it. Thus, it is exceptionally important to not discourage patient compliance by provoking skin-cleanser associated irritation.

Third, the patient may not properly understand the clinical role of topical tretinoin. The patient may thus use the topical tretinoin in lieu of, rather than in addition to, an acceptable skin cleanser. Failing to properly cleanse the skin, however, may in fact worsen the underlying acne vulgaris which the tretinoin is intended to treat.

As we discuss above, it may be possible to simply advise the patient to use trial-and-error to test various skin moisturizers to identify a skin moisturizer which does not exacerbate tretinoin-associated skin irritation. This approach, however, entails several shortcomings.

First, as with skin cleansers, a trial-and-error process will very likely force the patient to use several moisturizers which may exacerbate the tretinoin-associated skin irritation. This risk is especially great with the skin moisturizer, because unlike a skin cleanser (which is washed off of the skin), a moisturizer will remain on the skin surface for an extended period of time, and may be physically mixed with the tretionoin topical. This increases the risk that a component of the skin cream will exacerbate tretinoin-associated skin irritation, or will react with a component of the topical tretinoin to create an irritating product.

Second, as with skin cleansers, this risk is especially great where, as here, the benefits of the medication are not seen until after some delay.

Third, the patient may not properly understand the clinical role of topical tretinoin. A common misconception is that acne vulgaris is similar to dermal fungal infections (e.g., athlete's foot) in being caused by or associated with skin which is too damp. Thus, acne vulgaris patients may avoid using skin moisturizer at all, believing that skin moisturizer will cause acne vulgaris. Properly moistening the skin with a correctly-formulated (non-greasy) moisturizer, however, minimizes tretinoin-related skin irritation and, we believe, improves both patient therapeutic compliance and clinical outcomes.

Packaging

We prefer the tretinoin, the cleanser and the moisturizer to be packed together in a box. Other suitable packaging may, of course, be used. For example, one could provide a shrink-wrapped collection of three boxes; one box for each of tretinoin, moisturizer and cleanser. Alternatively, one could provide the three aforementioned components in tubes, and provide the various tubes in a plastic or metal display rack. One of skill in the art may readily design attractive alternatives; we thus use the term “packaging” in our claims to encompass everything which is included in the Federal Food, Drug & Cosmetic Act definition of “labeling.”

We claim:

1. A kit for treatment of acne vulgaris, the kit comprising:
a. tretinoin in a formulation acceptable for topical use, the formulation containing an amount of tretinoin effective for topical use to treat acne vulgaris;
b. a non-soap skin cleanser having a formulation which avoids exacerbating tretinoin-associated skin irritation with tretinoin; and
c. a non-greasy skin moisturizer having a formulation which avoids exacerbating tretinoin-associated skin irritation with tretinoin; and
d. packaging presenting said tretinoin, said skin cleanser and said skin moisturizer to a user for use together.

2. The kit of claim 1, said amount of tretinoin effective for topical use to treat acne vulgaris being a concentration of from about 0.1% to about 0.25% of the total formulation.

3. The kit of claim 1, wherein said non-soap skin cleanser includes green tea (Camellia sinensis) extract.

4. The kit of claim 1, wherein said non-greasy skin moisturizer includes a topical anti-irritant component.

5. The kit of claim 4, said topical anti-irritant component selected from the group consisting of: Aloe barbadensis leaf juice (aloe vera gel); glycerine, green tea (Camellia sinensis) extract; acetyl dipeptide-1 cetyl ester; and bisabolol.

6. The kit of claim 5, said topical anti-irritant component comprising Aloe barbadensis leaf juice (aloe vera gel), glycerine, green tea (Camellia sinensis) extract, acetyl dipeptide-1 cetyl ester and bisabolol.

7. The kit of claim 1, wherein
a. said amount of tretinoin effective for topical use to treat acne vulgaris being a concentration of from about 0.1% to about 0.25% of the total formulation;
b. said non-soap skin cleanser includes green tea (Camellia sinensis) extract; and
c. said non-greasy skin moisturizer includes a topical anti-irritant component selected from the group consisting of: Aloe barbadensis leaf juice (aloe vera gel);
glycerine, green tea (Camellia sinensis) extract; acetyl dipeptide-1 cetyl ester; and bisabolol.

8. A method for the treatment of acne vulgaris, the method comprising:
a. providing to a patient in need thereof tretinoin in a formulation acceptable for topical use, the formulation containing an amount of tretinoin effective for topical use to treat acne vulgaris;
b. providing to said patient a non-soap skin cleanser having a formulation which avoids exacerbating tretinoin-associated skin irritation with tretinoin; and
c. providing to said patient a non-greasy skin moisturizer having a formulation which avoids exacerbating tretinoin-associated skin irritation with tretinoin.

9. The method of claim 8, said amount of tretinoin effective for topical use to treat acne vulgaris being a concentration of from about 0.1% to about 0.25% of the total formulation.

10. The method of claim 8, wherein said non-soap skin cleanser includes green tea (Camellia sinensis) extract.

11. method kit of claim 8, wherein said non-greasy skin moisturizer includes a topical anti-irritant component.

12. The method of claim 11, said topical anti-irritant component selected from the group consisting of: Aloe barbadensis leaf juice (aloe vera gel); glycerine, green tea (Camellia sinensis) extract; acetyl dipeptide-1 cetyl ester; and bisabolol.

13. The method of claim 12, said topical anti-irritant component comprising Aloe barbadensis leaf juice (aloe vera gel), glycerine, green tea (Camellia sinensis) extract, acetyl dipeptide-1 cetyl ester and bisabolol.

14. The method of claim 8, wherein
a. said amount of tretinoin effective for topical use to treat acne vulgaris being a concentration of from about 0.1% to about 0.25% of the total formulation;
b. said non-soap skin cleanser includes green tea (Camellia sinensis) extract; and
c. said non-greasy skin moisturizer includes a topical anti-irritant component selected from the group consisting of: Aloe barbadensis leaf juice (aloe vera gel); glycerine, green tea (Camellia sinesis) extract; acetyl dipeptide-1 cetyl ester; and bisabolol.
